(19) United States
(12) Patent Application Publication
(10) Pub. No.: US 2021/0361473 A1
(43) Pub. Date: Nov. 25, 2021

(54) Battery Powered Heating Pad for the Breast

(71) Applicant: Laura C. Crowe, Danielsville, GA (US)

(72) Inventor: Laura C. Crowe, Danielsville, GA (US)

(21) Appl. No.: 16/882,009

(22) Filed: May 22, 2020

(51) Int. Cl. A61F 7/00; H01M 2/10; H05B 1/02; H05B 3/34; H02J 7/00

(52) U.S. Cl. CPC A61F 7/007; H01M 2/1066; H05B 1/0272; H05B 3/342; A41C 3/04; H01M 2220/30; A61F 2007/0078; A61F 2007/0086; A61F 2007/0021; H02J 7/0045

(57) ABSTRACT

A battery powered heating pad for the breast for lactating women. The battery powered heating pad for the breast generally includes a battery powered (or electrically-powered) heating pad for the breast. The heating pad includes a U-shaped body sized and shaped to be removably positioned within a pocket in a heating pad holder for attachment to a maternity bra. The heating pad also includes a U-shaped heating element positioned in alignment with the U-shaped body to provide heat at the outer surface of the U-shaped body. A power unit is also adapted to fit within the body, and is electrically connected to the heating element to provide electrical power to it. An open portion of the U-shaped body allows access to a woman's nipple without removing the electrically-powered heating pad.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND

### Field

Example embodiments in general relate to a battery powered heating pad for the breast for keeping the breasts of a lactating woman warm while still allowing for access to breastfeeding or pumping without removing or disconnecting the heating pad.

### Related Art

Any discussion of the related art throughout the specification should in no way be considered as an admission that such related art is widely known or forms part of common general knowledge in the field.

Some heating pads require heating in a microwave oven, and thus introduce a time delay, and cannot always be used, such as when there is no access to a microwave oven. In addition, microwave ovens may have cleanliness problems. Other devices may require cords and external batteries, or may be chemically-activated and suitable only for limited uses.

### SUMMARY

An example embodiment is directed to a Battery Powered (or electrically-powered) Heating Pad for the Breast. The Heating Pad includes a U-shaped body comprising an outer surface, the U-shaped body sized and shaped to be removably positioned within a pocket in a heating pad holder, the heating pad holder adapted for attachment to a maternity bra; a heating element adapted to fit within the U-shaped body, the heating element having a U shape and being positioned in alignment with the U-shaped body such that the heating element provides heat at the outer surface of the U-shaped body; and a power unit adapted to fit within the U-shaped body, the power unit electrically connected to the heating element to provide electrical power to the heating element. An open portion of the U-shaped body allows access to a woman's nipple without removing the electrically-powered heating pad.

In an example embodiment, the U-shaped body may be substantially flat in one dimension, to lay flat against the breast, although it may also be cup-shaped to follow the contour of the breast, and further, the outer surface of the U-shaped body is comprised of a heat conducting material. The U-shaped body may further be comprised of an electrically insulating material.

Some example embodiments may further comprise a power switch operatively connected to the power unit, wherein the power switch is usable to cause the power unit to supply power to the heating element. The power unit may comprise a battery, which may be a rechargeable battery. The power unit may further comprise a control circuit that controls application of power from the battery to the heating element. The control circuit may be operatively connected to a power switch, and the power switch may be usable to cause the power unit to supply power to the heating element.

In embodiments wherein the power unit comprises a rechargeable battery and a control circuit, the control circuit may be usable to control application of power from the battery to the heating element, and may also be usable to control a charging current for the rechargeable battery. The heating pad may further comprise a charging port, and the control circuit can control (e.g., with modulation of the current level, or by switching the current on and oft or any combination of such control) a charging current between the charging port and the rechargeable battery.

Some example embodiments may include a method of using an electrically-powered heating pad, comprising the steps of charging a rechargeable battery by connecting a power source to the charging port, inserting the electrically-powered heating pad into the heating pad holder, and pressing the power switch to cause the control circuit to supply power to the heating element. These steps can of course occur in different orders, and at different times. For example, the charging step can take place immediately before, or well before, the power is switched on to begin heating.

Another example embodiment of an electrically-powered heating pad for heating the breast of a lactating woman may comprise a disk-shaped body comprising an outer surface, the disk-shaped body sized and shaped to be removably positioned in a garment next to a woman's breast; a heating element adapted to fit within the disk-shaped body, the heating element having a disk shape and being positioned in alignment with the disk-shaped body such that the heating element provides heat at the outer surface of the disk-shaped body; and a power unit adapted to fit within the disk-shaped body, the power unit electrically connected to the heating element to provide electrical power to the heating element.

Some example embodiments of the disk-shaped heating pad may further comprise a power switch operatively connected to the power unit, wherein the power switch is usable to cause the power unit to supply power to the heating element. The power unit may comprise a battery, which may be a rechargeable battery. The power unit may further comprise a control circuit that controls application of power from the battery to the heating element. The control circuit may be operatively connected to a power switch, as described generally above, and the power switch may be usable to cause the power unit to supply power to the heating element.

As with other embodiments, the electrically-powered heating pad may comprise a disk-shaped body that is substantially flat. The body may further comprise an outer surface that is a heat conducting material, which may also be electrically insulating.

There has thus been outlined, rather broadly, some of the embodiments of the battery powered heating pad for the breast in order that the detailed description thereof may be better understood, and in order that the present contribution to the art may be better appreciated. There are additional embodiments of the battery powered heating pad for the breast that will be described hereinafter and that will form the subject matter of the claims appended hereto. In this respect, before explaining at least one embodiment of the battery powered heating pad for the breast in detail, it is to be understood that the battery powered heating pad for the breast is not limited in its application to the details of construction or to the arrangements of the components set forth in the following description or illustrated in the drawings. The battery powered heating pad for the breast is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a front perspective view of a pair of battery powered heating pads in accordance with an example embodiment.

FIG. 2 is another front perspective view of a pair of battery powered heating pads in accordance with an example embodiment.

FIG. 3 is a rear perspective view of cloth sleeve for holding a pair of battery powered heating pads in accordance with an example embodiment.

FIG. 4 is an exploded perspective view of a pair of battery powered heating pads in accordance with an example embodiment.

FIG. 5 is a front perspective view of a pair of battery powered heating pads in accordance with an example embodiment.

FIG. 6A is a front view of a battery powered heating pad in accordance with an example embodiment.

FIG. 6B is a rear view of a battery powered heating pad in accordance with an example embodiment.

FIG. 7 is a sectional view of a battery powered heating pad in accordance with an example embodiment.

FIG. 8 is a perspective view of a pair of battery powered heating pads being charged in accordance with an example embodiment.

FIG. 9 is a front view of a block diagram of a battery powered heating pad in accordance with an example embodiment.

FIG. 10 is an exploded perspective view of a pair of battery powered heating pads in accordance with another example embodiment.

FIG. 11 is a front perspective view of a pair of battery powered heating pads in accordance with another example embodiment.

FIG. 12 is a sectional view of a battery powered heating pad in accordance with another example embodiment.

### DETAILED DESCRIPTION

### A. Overview.

An example battery- or electrically-powered heating pad 10 for the breast generally comprises a U-shaped body 17 having an outer surface 13, the U-shaped body sized and shaped to be removably positioned within a pocket 33 in a heating pad holder 30, the heating pad holder 30 adapted for attachment to a maternity bra 50.

The holder, or cloth sleeve, 30, may be made of cloth or other suitable material, and may comprise a front outer surface 38 and a rear outer surface 39, the latter of which will generally be facing, or touching, a bra or maternity bra 50 of a user. Accordingly, the cloth sleeve 30 may be made of thin, compliant material, selected for both comfort and efficient heat transfer.

Also included is a heating element 14 adapted (e.g., sized and shaped) to fit within the U-shaped body 17, the heating element 14 also having a U shape and being positioned in alignment with the U-shaped body 17 such that the heating element 14 provides heat at the outer surface 13a, 13b of the U-shaped body 17. The heating pad may also include a power unit 15 adapted to fit within the U-shaped body 17, the power unit 15 electrically connected to the heating element 14 to provide electrical power to the heating element 14. An open portion 22 of the U-shaped body 17 allows access to a woman's nipple without removing the electrically-powered heating pad 10 from a holder, such as a cloth sleeve. The cloth sleeve or other holding device may be designed for attachment to a maternity bra 50.

The U-shaped heating pad body 17 may be substantially flat (e.g., thin), and may also have a cup shape to allow it to have good contact with the breast, and further, the outer surfaces 13a, 13b of the U-shaped body 17 may be comprised of a heat conducting material. The U-shaped body 17 may further be comprised of an electrically insulating material. For example, the outer surfaces 13a, 13b may be made of washable or water-resistance fabric, treated fabric, and may be woven or non-woven. The heating element 14 may be made so that it is close to one or both surfaces 13a and 13b, and particularly close to rear or back surface 13b, which may be the outer surface of the heating pad 10 that is closest to the breast when in use. For example, the surface 13b may be separated from the breast by only one or two layers of cloth, which may be designed and fabricated to be thin and heat conducting, to better conduct heat. The cloth layers may be or comprise the fabric of a cloth sleeve 30 that is made to be clipped or attached to a maternity bra 50, and the upper surface of the maternity bra 50 itself, just above the nipple.

Some example embodiments may further comprise a power switch or on/off switch 20 operatively connected to the power unit 15, wherein the power switch 20 is usable to cause the power unit 15 to supply power to the heating element 14. The power unit may comprise a battery 12, which may be a rechargeable battery, and may be a battery pack comprising more than one battery. The rechargeable batteries may be rechargeable alkaline, Ni-Cad, Lithium polymer, or other types. The power unit/batteries should have sufficient capacity to provide power to the heating element 14. The power unit 15 may further comprise a control circuit 16 that controls application of power from the battery 12 to the heating element 14. The control circuit 16 may be operatively connected to the power switch 20, and the power switch 20 may thus be usable to cause the power unit 15 to supply power to the heating element 14.

In embodiments wherein the power unit 15 comprises a rechargeable battery 12 and a control circuit 16, the control circuit 16 may be usable to control application of power from the battery 12 to the heating element 14, and may also be usable to control a charging current for the rechargeable battery 12. The heating pad 10 may further comprise a charging port 18, and the control circuit 16 can control a charging current between the charging port 18 and the rechargeable battery 12.

Another example battery- or electrically-powered heating pad 60 generally comprises a disk-shaped body 62 comprising an outer surface 64 (comprised, e.g., of front surface 64a and back surface 64b), the disk-shaped body 62 sized and shaped to be removably positioned within a pocket 33 in a heating pad holder 30, the heating pad holder 30 adapted for attachment to a maternity bra 50, such as with attachments 36. This embodiment may also include a heating element 14 adapted to fit within the disk-shaped body 62, the heating element 14 also having a disk shape and being positioned in alignment with the disk-shaped body 62 such that the heating element 14 provides heat at the outer surfaces 64a, 64b of the disk-shaped body 62. The device may further include a power unit 15 adapted to fit within the disk-shaped body 62, the power unit 15 electrically connected to the heating element 14 to provide electrical power to the heating element 14.

The disk-shaped body 62 may be substantially flat (e.g., may have a thin cross section), and may be cup-shaped to fit the breast and have good contact with it. Further, the outer surfaces 64a, 64b of the disk-shaped body 62 may be comprised of a heat conducting material. The disk-shaped body 62 may further be comprised of an electrically insulating material. For example, the outer surfaces 64a, 64b may be made of washable or water-resistance fabric, treated fabric, and may be woven or non-woven. The heating element 14 may be made so that it is close to one or both surfaces 64a and 64b, and particularly, close to rear or back surface 64b, which may be the outer surface of the heating pad 60 that is closest to the breast when in use. For example, the surface 64b may be separated from the breast by only one or two layers of cloth, which may be designed and fabricated to be thin and thermally conductive to better transfer heat. The cloth layers may be or comprise the fabric of a cloth sleeve 30 that is made to be clipped or attached to a maternity bra 50, and the upper surface of the maternity bra 50 itself, just above the nipple.

The disk-shaped embodiment of the heating pad may advantageously be used without a cloth sleeve for holding the device, although such a sleeve/holder is an option. The disk-shaped heating pad may be inserted directly into the cup of a maternity bra or an ordinary bra to provide heat to the breast.

### B. Heating Pad.

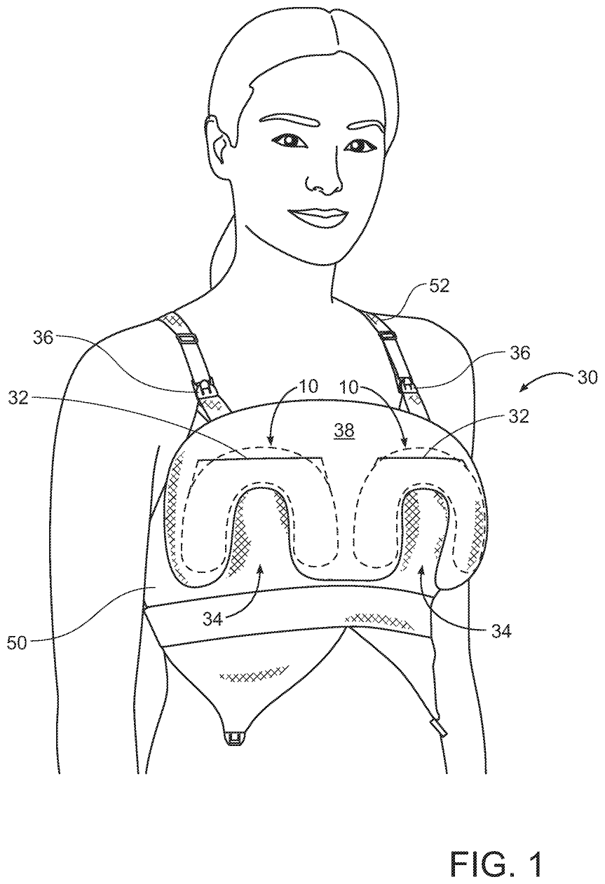

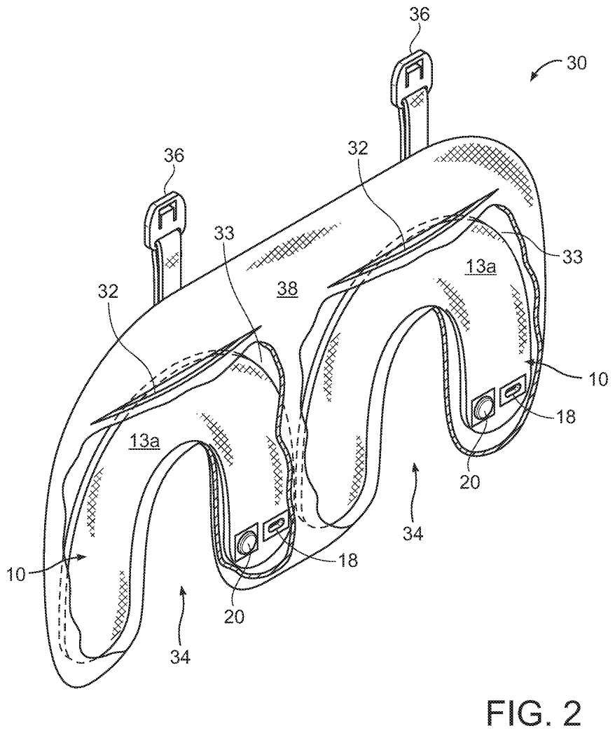

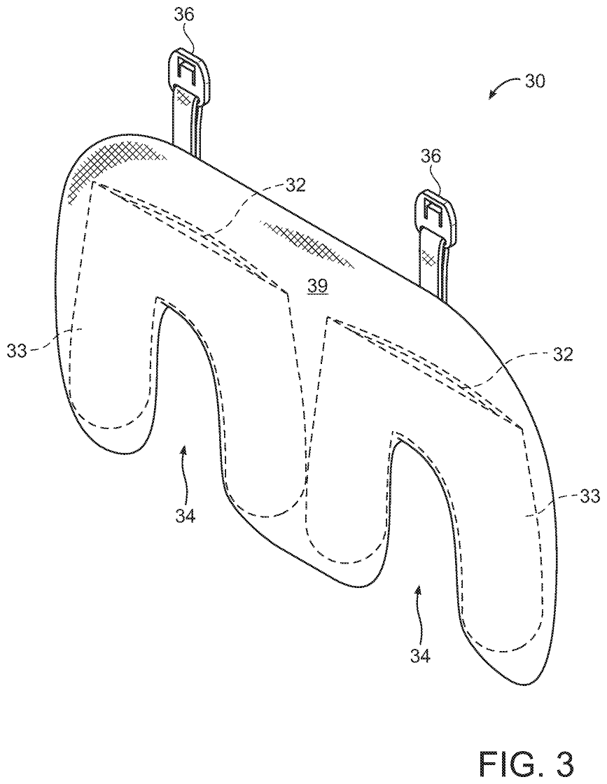

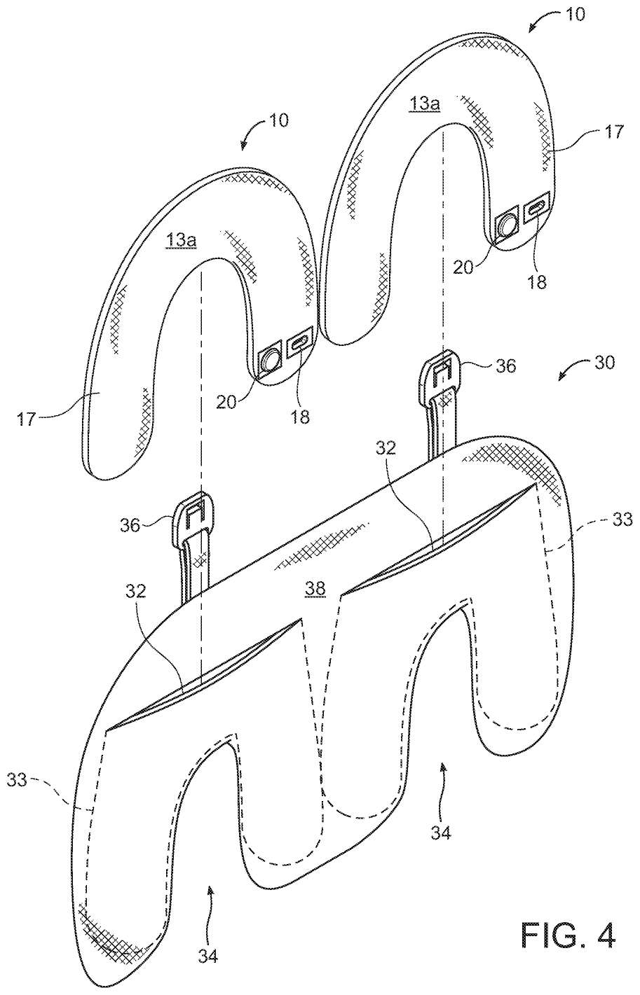

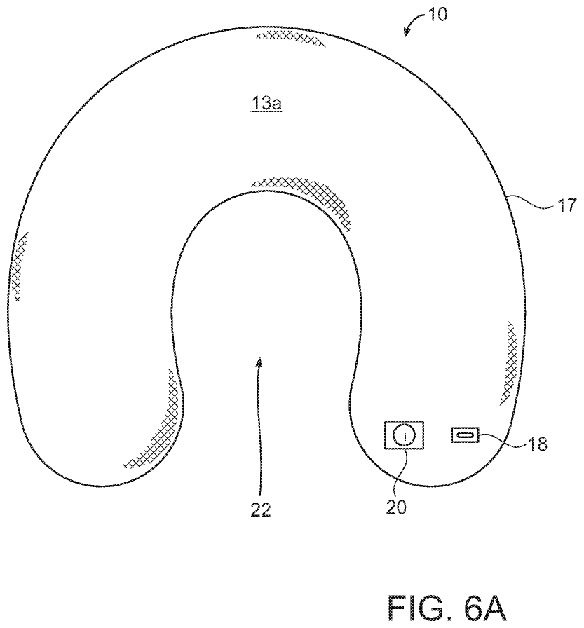

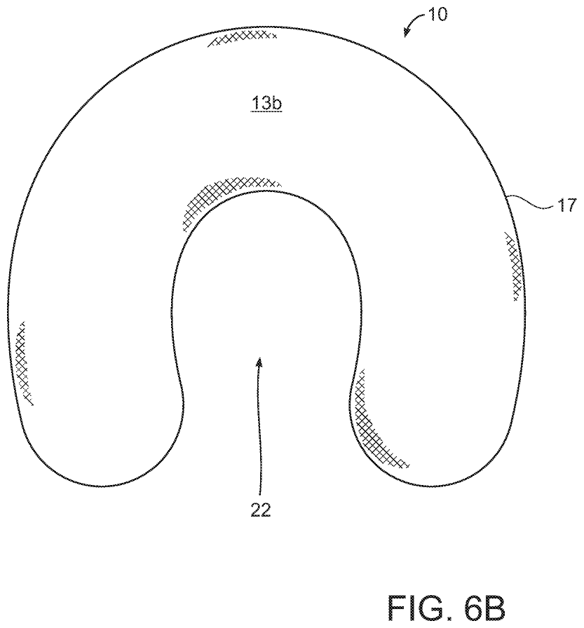

An example battery or electrically powered heating pad 10 may comprise a U-shaped body 17 as shown in FIGS. 1-2 and 4-8. The heating pad may comprise an outer surface 13, which includes a front outer surface 13a and a back or rear outer surface 13b. The U-shaped body 17 is generally sized and shaped to be held within a pocket 33 in a heating pad holder 30, which may also be referred to as a cloth sleeve 30, although it may be made of virtually any suitable material, although the material should of course be selected for comfort and heat transfer. The heating pad holder 30 (see FIGS. 3-4) is adapted for attachment to a maternity bra 50, and may be held in place by attachments 36, which may be or include clips designed to fit mating attachment elements on the maternity bra (not shown) or which may simply clip onto the straps 52 of the maternity bra 50 or other bra or garment. The holder or sleeve 30 also comprises a front outer surface 38, a rear outer surface 39, and openings 32 in communication with pockets 33, such that heating pads 10 or 60 can be inserted into the holder or sleeve 30. The openings 32 may be simple openings in form, or they may alternatively have closures, such as Velcro, snaps, buttons, etc.

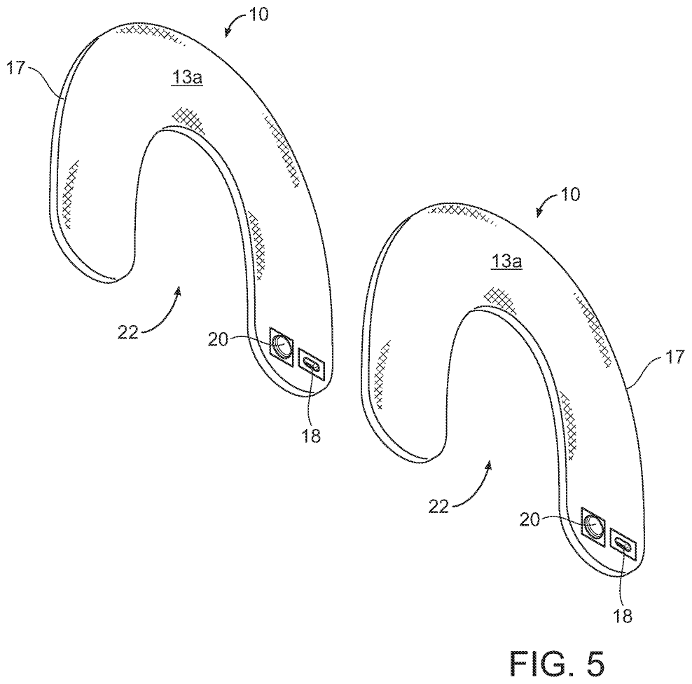

The U-shaped body 17 comprises a downward-facing opening or open portion 22, as shown in FIG. 5. This opening corresponds to an open portion 34 of holder 30 (see FIG. 3) to allow access to the flange of a breast pump, or to a nursing baby, so that the heating pad 10 can be used without removal during feeding or pumping.

The heating pad 10 may be designed and manufactured to be a complete, self-contained unit, so that it fits entirely within a pocket 33 of a cloth sleeve or holder 30, with no wires or other elements extending out of the pocket 33. Such placement is perhaps best shown, for example, in FIGS. 1, 2, and 4, while the cloth sleeve is shown in FIG. 3. Accordingly, the pad 10 may be slipped or inserted into the pocket 33 via opening 32 and left there during use, or until needed for later use.

As mentioned above, the heating pad 10 may also include a heating element 14 adapted to fit within the U-shaped body 17, the heating element 14 also having a U shape and being positioned and aligned with the U-shaped body 17 such that the heating element 14 provides heat at the outer surface 13a, 13b of the U-shaped body 17. The U-shaped body 17 may be substantially flat, and further, the outer surface 13a, 13b of the U-shaped body 17 may be comprised of a heat conducting material. The U-shaped body 17 may further be comprised of an electrically insulating material, such that the body 17 is in essence a self-contained envelope. For example, the outer surfaces 13a, 13b may be made of washable or water-resistance fabric or material, or treated fabric, and may be woven or non-woven.

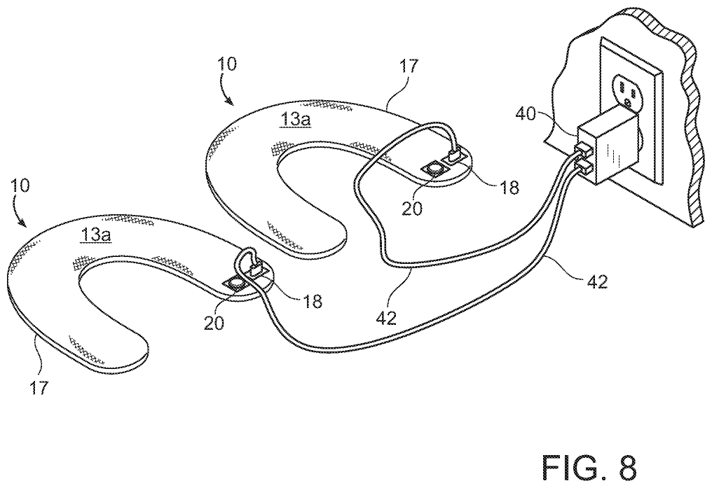

The heating pad 10 may also include a power unit 15 adapted to fit within the U-shaped body 17, the power unit 15 electrically connected to the heating element 14 to provide electrical power to the heating element 14. The power unit may comprise a battery 12, which may be a rechargeable battery, and may be a battery pack comprising more than one battery. The rechargeable batteries may be rechargeable alkaline, Ni-Cad, Lithium polymer, Nickel-Metal Hydride, Lithium-Ion, or other types. The power unit/batteries should have sufficient capacity to provide power to the heating element 14. The power unit 15 may further comprise a control circuit 16, and the control circuit 16 may be usable to control application of power from the battery 12 to the heating element 14, and may also be usable to control a charging current for the rechargeable battery 12. The charging current may be supplied through a charging port 18, and the control circuit 16 can control a charging current between the charging port 18 and the rechargeable battery 12. As shown in FIG. 8, the charging ports 18 may be conventional ports such as those used on mobile phones and other electronic devices, and thus may be charged using conventional charger 40 and readily-available cables 42.

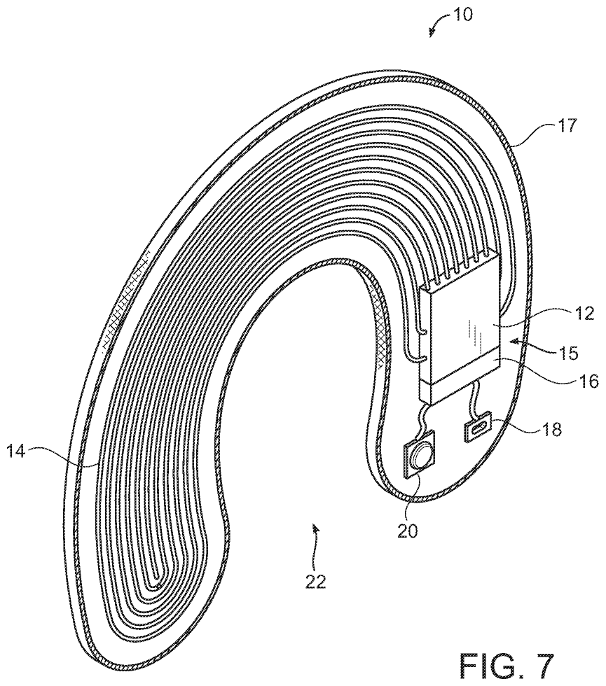

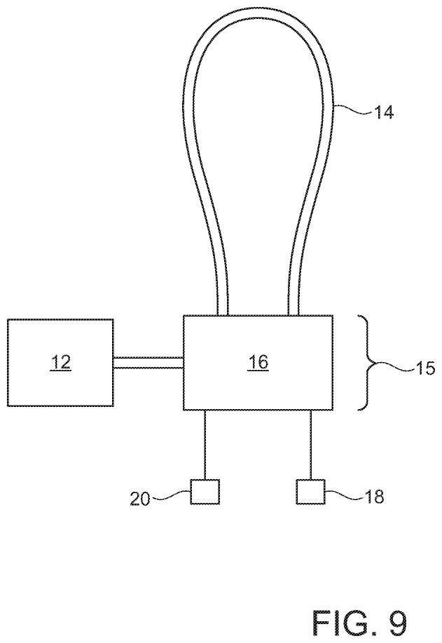

The control circuit 16 may be operatively connected to the charging port 18 and the power switch 20, and the power switch 20 may thus be usable to cause the power unit 15 to supply power to the heating element 14. The control circuit may include temperature control circuitry to maintain a given power level or closed-loop temperature of the heating element 14, which may be controlled using a temperature sensor, such as a thermistor or a thermocouple. The output of such a temperature sensor may also be applied to control circuit 16 so that temperature, as well as the input from switch 20, may be used to control the heating pad. The temperature set point may be preselected and integrated into the control circuit, or the heating pad may also include a temperature control, with a set point that is selectable by the user, such as with a linear control or predetermined levels (e.g., low, medium, and high). The control circuit 16 may thus include an output device, such as a power transistor or switch, that supplies power from one or more batteries to the heating element 14. A physical representation of these components is shown in FIG. 7, and a block diagram of the connections is shown in FIG. 9.

The power switch 20 provides an input to control circuit 16 to cause the power unit 15 to supply power to the heating element 14. The power may be supplied on a time-limited basis, or may simply stay on until the switch is used to turn the heating pad 10 off. The power switch 20 may be a membrane-type switch, and thus provide tactile feedback, so that it can be used even while the heating pad 10 is positioned within cloth sleeve 30, by allowing a user to feel and press the switch through the material of sleeve 30.

The heating element 14 may be made so that it is close to one or both surfaces 13a and 13b, and particularly rear or back surface 13b, which may be the outer surface of the heating pad 10 that is closest to the breast when in use. For example, the surface 13b may be separated from the breast by only one or two layers of cloth, which may be designed and fabricated to be thin to better conduct heat. The cloth layers may be or comprise the fabric of a cloth sleeve 30 that is made to be clipped or attached to a maternity bra 50, and the upper surface of the maternity bra 50 itself, just above the nipple.

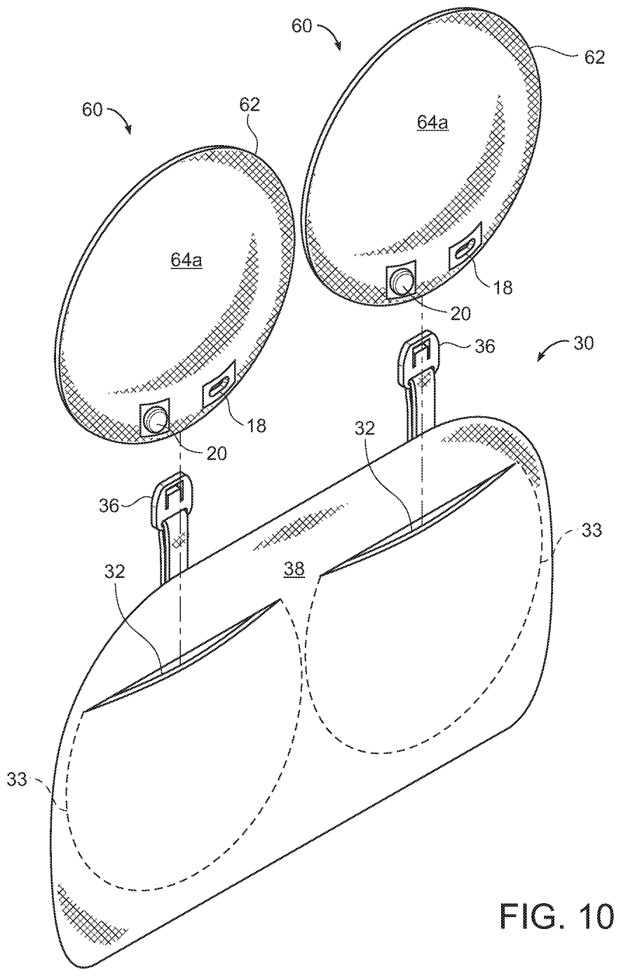

In addition to the substantially U-shaped embodiment of the heating pad 10 shown in FIGS. 1-2 and 4-9, the heating pad may also be made in a full-circle shape, represented in the figures as heating pad 60 (see FIGS. 10-12). As with other example embodiments, heating pads 60 may be sized and shaped for being positioned or held in cloth sleeve 30, which may have alternatively shaped pockets 33, as shown in FIG. 10, which in this instance may be generally circular. As with the U-shaped embodiment, the pockets 33 each have an opening 32 at the upper part of the pocket, into which the pads 60 can be inserted. The pads 60 of this embodiment comprise a circular shaped body 62, which may be insertable into cloth sleeve 30. In addition to being positionable in a cloth sleeve or holder 30, heating pads 60 may also be placed in and used in other garments, such as a non-maternity bra, for which use the pads 60 may be placed inside the bra or other garment, such that the outer surface 64b is in direct contact with the breast.

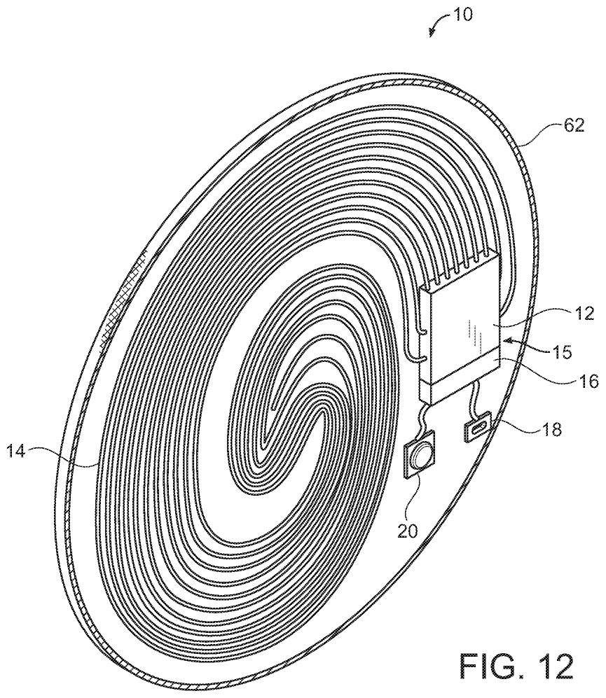

The heating pad 60 may also include a heating element 14 adapted (e.g., sized and shaped) to fit within the circular body 62, the heating element 14 in this alternative embodiment having a circular shape (as shown in FIG. 12) and being positioned and aligned with the circular-shaped body 62 such that the heating element 14 provides heat at the outer surfaces 64a, 64b of the body 62. The generally circular body 62 may be substantially flat, and further, the outer surfaces 64a, 64b of the body 62 may be comprised of a heat conducting material. The body 62 may further be comprised of an electrically insulating material. For example, the outer surfaces 64a, 64b may be made of washable or water-resistance fabric, treated fabric, and may be woven or non-woven.

As also shown in FIG. 12, the heating pads 60, as with other example embodiments, may also include a power unit 15 adapted to fit within the body 62, the power unit 15 electrically connected to the heating element 14 to provide electrical power to the heating element 14. The power unit may comprise a battery 12, which may be a rechargeable or conventional battery, as discussed above regarding the U-shaped embodiment. The power unit 15 may further comprise a control circuit 16, and the control circuit 16 may be usable to control application of power from the battery 12 to the heating element 14, and may also be usable to control a charging current for the rechargeable battery 12. The charging current may be supplied through a charging port 18, and the control circuit 16 can control a charging current between the charging port 18 and the rechargeable battery 12.

The control circuit 16, charging port 18 and power switch 20 as used in the circular embodiment of heating pad 60 typically function as described above in detail with regard to the U-shaped embodiment of the heating pad.

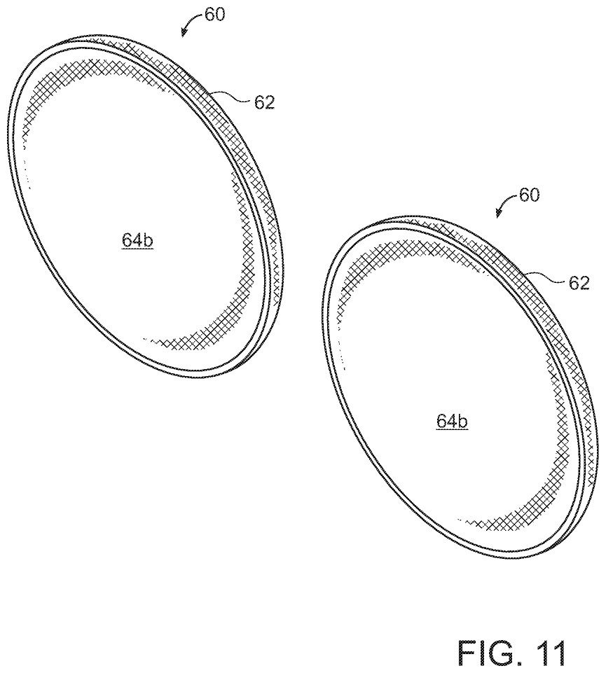

As shown in FIGS. 10 and 11, the circular embodiment of heating pads 60 include a front outer surface 64a, and a rear or back outer surface 64b, which will generally be closest to the breast. Thus, as with other example embodiments, the heating element 14 (see FIG. 12) may be placed or positioned such that heat is readily transferred to back surface 64b.

### C. Operation of Preferred Embodiment.

A battery-powered heating pad has advantages not present in some other heating devices. For example, the example embodiments do not need to be heated before use, such as in an unsanitary microwave oven. Working women, for example, need heating devices that can be used and available quickly, without cords (although a corded version of the example embodiments is possible, and the embodiments herein are also capable of being charged while in use).

In use, an example embodiment may have a U-shape that allows access to the nipple with a breast pump or a baby. As shown in FIG. 1, heating pads 10 can be placed pockets 33 of a cloth sleeve 30 and wrapped around the pumping flange of a breast pump (not shown). Thus, the heating pads are sized and shaped so that they can be used during pumping or breastfeeding without first removing them or turning the heat off. The cloth sleeve, with its openings 34, is also designed for such accommodation, and it has pockets 33 that are shaped to receive and hold heating pads 10 (or alternative embodiment heating pads 60), and to efficiently transfer heat to the breasts of a user.

Typically, the heating pads 10 or 60 will be charged (see FIG. 8) after a previous use, or before use, so that when they are needed, they are fully charged. The power switch 20 can be used to activate heating pads 10 either before or after the pads are placed in a sleeve 30 or other garment, after which control circuit 16 will cause electrical power to be applied to heating elements 14. The power switch may initiate a timer that turns supplies power to the heating elements 14 for a fixed time period, or the switch may be used to manually turn off the power unit 15.

Thus activated, the heating pads heat the breasts, which helps breastfeeding women let down more milk, as the heat promotes more blood flow to the breasts. Lactating women can also wear heating pads 10 or 60 during the day (e.g., when not breast feeding or pumping) to keep the breasts and nipples warm to decrease vasospasms.

Unless otherwise defined, all technical and scientific terms used herein have the same meaning as commonly understood by one of ordinary skill in the art to which this invention belongs. Although methods and materials similar to or equivalent to those described herein can be used in the practice or testing of the battery powered heating pad for the breast, suitable methods and materials are described above. All publications, patent applications, patents, and other references mentioned herein are incorporated by reference in their entirety to the extent allowed by applicable law and regulations. The battery powered heating pad for the breast may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. An electrically-powered heating pad for heating a breast while allowing for breastfeeding or pumping, comprising:
a U-shaped body comprising an outer surface, the U-shaped body sized and shaped to be removably positioned within a pocket in a heating pad holder, the heating pad holder adapted for attachment to a maternity bra;
a heating element adapted to fit within the U-shaped body, the heating element having a U shape and being positioned in alignment with the U-shaped body such that the heating element provides heat at the outer surface of the U-shaped body; and
a power unit adapted to fit within the U-shaped body, the power unit electrically connected to the heating element to provide electrical power to the heating element;
wherein an open portion of the U-shaped body allows access to a woman's nipple without removing the electrically-powered heating pad.

2. The electrically-powered heating pad of claim 1, wherein the U-shaped body is substantially flat.

3. The electrically-powered heating pad of claim 1, wherein the outer surface of the U-shaped body is comprised of a heat conducting material.

4. The electrically-powered heating pad of claim 1, further comprising a power switch operatively connected to the power unit, wherein the power switch is usable to cause the power unit to supply power to the heating element.

5. The electrically-powered heating pad of claim 1, wherein the power unit comprises a battery.

6. The electrically-powered heating pad of claim 5, wherein the battery is rechargeable.

7. The electrically-powered heating pad of claim 5, wherein the power unit further comprises a control circuit that controls application of power from the battery to the heating element.

8. The electrically-powered heating pad of claim 1, wherein the power unit comprises a control circuit that controls an application of power from the battery to the heating element.

9. The electrically-powered heating pad of claim 8, wherein the control circuit is operatively connected to a power switch, and wherein the power switch is usable to cause the power unit to supply power to the heating element.

10. The electrically-powered heating pad of claim 1, wherein the power unit comprises a rechargeable battery and a control circuit, the control circuit usable to control application of power from the battery to the heating element, and to control a charging current for the rechargeable battery.

11. The electrically-powered heating pad of claim 10, further comprising a charging port, wherein the control circuit controls a charging current between the charging port and the rechargeable battery.

12. A method of using the electrically-powered heating pad of claim 10, comprising the steps of:
charging the rechargeable battery by connecting a power source to the charging port;
inserting the electrically-powered heating pad into the heating pad holder; and
pressing the power switch to cause the control circuit to supply power to the heating element.

13. An electrically-powered heating pad for heating the breast of a lactating woman, comprising:
a disk-shaped body comprising an outer surface, the disk-shaped body sized and shaped to be removably positioned in a garment next to a woman's breast;
a heating element adapted to fit within the disk-shaped body, the heating element having a disk shape and being positioned in alignment with the disk-shaped body such that the heating element provides heat at the outer surface of the disk-shaped body; and
a power unit adapted to fit within the disk-shaped body, the power unit electrically connected to the heating element to provide electrical power to the heating element.

14. The electrically-powered heating pad of claim 13, wherein the disk-shaped body is substantially flat.

15. The electrically-powered heating pad of claim 13, wherein the outer surface of the disk-shaped body comprises a heat conducting material.

16. The electrically-powered heating pad of claim 13, further comprising a power switch operatively connected to the power unit, wherein the power switch is usable to cause the power unit to supply power to the heating element.

17. The electrically-powered heating pad of claim 13, wherein the power unit comprises a battery.

18. The electrically-powered heating pad of claim 17, wherein the battery is rechargeable.

19. The electrically-powered heating pad of claim 17, wherein the power unit further comprises a control circuit that controls application of power from the battery to the heating element.

20. An electrically-powered heating pad for heating a breast while allowing for breastfeeding or pumping, comprising:
a U-shaped body comprising an outer surface comprising a heat-conducting material, the U-shaped body being substantially flat and sized and shaped to be removably positioned within a pocket in a heating pad holder, the heating pad holder adapted for attachment to a maternity bra;
a heating element adapted to fit within the U-shaped body, the heating element having a U shape and being positioned in alignment with the U-shaped body such that the heating element provides heat at the outer surface of the U-shaped body; and
a power unit comprising a rechargeable battery and a control circuit, the power unit adapted to fit entirely within the U-shaped body, the power unit electrically connected to the heating element to provide electrical power to the heating element;
a power switch operatively connected to the control circuit; and
a charging port operatively connected to the power unit;
wherein the control circuit controls an application of power from the rechargeable battery to the heating element;
wherein the control circuit controls a charging current between the charging port and the rechargeable battery;
wherein the power switch is usable to cause the power unit to supply power to the heating element; and
wherein an open portion of the U-shaped body allows access to a woman's nipple without removing the electrically-powered heating pad.
